(19) United States
(12) Patent Application Publication
(10) Pub. No.: US 2005/0244352 A1
(43) Pub. Date: Nov. 3, 2005

(54) Cosmetic composition of the water-in-oil emulsion type comprising a deodorant active salt and a polyolefin-derived emulsifier comprising at least one polar part

(76) Inventors: Cyril Lemoine, Puiseux-en-France (FR); Emmanuelle Lebon-Hipolite, Abbecourt (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/105,501

(22) Filed: Apr. 14, 2005

Related U.S. Application Data
(60) Provisional application No. 60/580,102, filed on Jun. 17, 2004.

(30) Foreign Application Priority Data
Apr. 15, 2004 (FR) 04 03941

(51) Int. Cl. A61K 7/32; A61K 7/34

(52) U.S. Cl. 424/66

(57) ABSTRACT

A cosmetic composition comprising, (A) at least one deodorant active salt; (B) at least one polyolefin-derived emulsifying oligomer or polymer comprising at least one polar part, wherein the composition is in the form of a water-in-oil emulsion. A cosmetic process for the treating perspiration comprising applying, to the surface of the skin, an effective amount of at least one composition. A cosmetic process for treating body odors related to perspiration (for example, axillary odors or odors of the feet), comprising applying, to the surface of the skin, an effective amount of the composition. Use of at least one polyolefin-derived emulsifying oligomer or polymer comprising at least one polar part for stabilizing a water-in-oil emulsion comprising at least one deodorant or antiperspirant active salt.

This application claims benefit of U.S. Provisional Application No. 60/580,102, filed Jun. 17, 2004, the contents of which are incorporated herein by reference. This application also claims benefit of priority under 35 U.S.C. § 119 to French Patent Application No. FR 04/03941 filed Apr. 15, 2004, the contents of which are also incorporated by reference.

Disclosed herein is a cosmetic composition in the form of a water-in-oil emulsion, comprising:

(A) at least one deodorant active salt and

(B) at least one polyolefin-derived emulsifying oligomer or polymer comprising at least one polar part.

Further disclosed herein is a cosmetic process for treating perspiration comprising applying, to the surface of the skin, an effective amount of at least one cosmetic composition as described above.

In the cosmetics field, it is well known to use, in topical application, deodorant products comprising active substances, such as antiperspirants, bactericides, and odor absorbers to reduce, indeed even eliminate, generally unpleasant axillary odors and/or to absorb sweat.

Antiperspirant substances, which have the effect of reducing or eliminating the flow of sweat, generally comprise aluminium salts.

Many different types of antiperspirant compositions have been described in the literature and have appeared on the market in many forms, such as gels, sticks, creams, roll-ons, or aerosols.

Deodorant or antiperspirant active salts, for example, water-soluble antiperspirant salts, may be difficult to formulate in emulsion vehicles comprising an aqueous phase. The acidity, such as that of the antiperspirant active principles, and the high level of salts mean that the majority of the emulsions comprising them may not be stable or may be stable with difficulty. Furthermore, deodorant active principles in the form of salts may often be tacky during drying on the skin.

The deodorant or antiperspirant emulsions conventionally used are oil-in-water emulsions comprising an oily phase dispersed in a continuous aqueous phase and generally comprising nonionic emulsifiers. This type of emulsion may have the disadvantage of having long drying times and of contributing a tacky feel during drying on the skin.

The water-in-oil emulsions comprising an aqueous phase dispersed in a continuous oily phase are a minority in the field of deodorants and antiperspirants.

Water-in-oil deodorant or antiperspirant emulsions of water-in-silicone type are known. These emulsions, comprising silicone surfactants and silicone oils, are the major constituents of the oil phase. The feel of these emulsions remains characteristic of silicones.

Water-in-oil antiperspirant compositions rich in aqueous phase (at least 70%) and comprising a nonionic emulsifier of the alkylated derivative of polyethylene glycol type (for example, Polyglyceryl-3 Isostearate) or of the alkylated derivative of polyglycerol type (for example: PEG-30 Dipolyhydroxystearate) are described, for example, in Patent Application No. DE 10210461. These emulsions may have the disadvantage of being prepared under hot conditions (75° C.).

Antiperspirant aerosols in the form of a water-in-oil emulsion comprising a nonionic emulsifier of polyglycerol-4 oleate are described in Patent Application No. CA 1076030.

Antiperspirant aerosols in the form of a water-in-oil emulsion comprising a nonionic emulsifier, for example, sorbitan sesquioleate, and a mixture of oil comprising a hydrocarbon and an emollient oil are described, for example, in Patent Application No. GB 2 113 706.

These water-in-oil emulsions of the prior art exhibit at least one of the following disadvantages:

they may not contribute freshness to the application,

they may leave a greasy film on the skin,

they may have problems of stability in the presence of deodorant active principles in the salt form, and

they may have a tendency to reduce or inhibit the activity of the deodorant active salts.

There thus exists a need to try to find novel deodorant or antiperspirant formulations in the form of a water-in-oil emulsion comprising at least one deodorant or antiperspirant active salt which does not exhibit at least some of the disadvantages discussed above.

The present inventors have discovered, surprisingly and unexpectedly, that the use of at least one polyolefin-derived emulsifying oligomer or polymer comprising at least one polar part in a water-in-oil emulsion comprising at least one active salt chosen from deodorant and antiperspirant active salts may make it possible to obtain deodorant or antiperspirant products which may be stable over time even with high levels of the at least one active salt chosen from deodorant and antiperspirant active salts. These products may exhibit good effectiveness and produce, after rapid drying, a fresh feeling on the skin on application, produce a nontacky or not very tacky effect, do not produce a greasy film and/or do not produce a whitening phenomenon.

Disclosed herein is thus a cosmetic composition comprising,

(A) at least one active salt chosen from deodorant and antiperspirant active salts and

(B) at least one polyolefin-derived emulsifying oligomer or polymer comprising at least one polar part,

wherein the composition is in the form of a water-in-oil emulsion.

Further disclosed herein is a cosmetic process for treating perspiration comprising applying, to the surface of the skin, an effective amount of the above-described composition.

The treatment of perspiration can comprise both reducing or eliminating the unpleasant odor brought about by sweat and reducing or eliminating the feeling of dampness produced by sweat.

Further disclosed herein is a cosmetic process for treating body odors related to perspiration (for example, axillary odors or odors of the feet), comprising applying, to the surface of the skin, an effective amount of at least one composition as described above.

Even further disclosed herein is the use of at least one polyolefin-derived emulsifying oligomer or polymer comprising at least one polar part for stabilizing a water-in-oil emulsion comprising at least one active salt chosen from deodorant and antiperspirant active salts.

Even further disclosed herein is the use of at least one polyolefin-derived emulsifying oligomer or polymer comprising at least one polar part in a cosmetic composition in the form of a water-in-oil emulsion comprising at least one active salt chosen from deodorant and antiperspirant active salts, wherein the at least one polyolefin-derived emulsifying oligomer or polymer is present in an amount sufficient to improve at least one sensory property on the skin after application of the composition.

As used herein, the term “at least one sensory property on the skin” is understood to mean the sensation of freshness, the nontacky effect and/or the nongreasy effect.

As used herein, the term “water-in-oil emulsion” is understood to mean any composition comprising an aqueous phase dispersed in a continuous oily phase.

As used herein, the term “deodorant active principle” is understood to mean any substance capable of reducing or eliminating the flow of sweat and/or of absorbing human sweat and/or of masking, absorbing, improving or reducing the unpleasant odor resulting from the decomposition of human sweat by bacteria.

As used herein, the term “antiperspirant active principle” is understood to mean any substance capable of reducing the flow of sweat and/or of absorbing human sweat.

The polyolefin-derived emulsifying oligomers and/or polymers which can be used in the composition disclosed herein are known in other fields. For example, such oligomers or polymers are disclosed in U.S. patent nos. U.S. Pat. No. 5,129,972 and U.S. Pat. No. 4,919,179 as stabilizers for explosive emulsions. As another example, these polyolefin-derived emulsifying oligomers and polymers are known as stabilizers for fertilizing compositions (see U.S. patent nos. U.S. Pat. No. 5,518,517 and U.S. Pat. No. 5,858,055) for the purpose of obtaining controlled release of the fertilizing substances.

The at least one polyolefin-derived emulsifying oligomer or polymer used in the composition disclosed herein comprises at least one nonpolar polyolefinic part and at least one polar part. The at least one polyolefin-derived emulsifying oligomer or polymer can have a structure of block or comb type.

The at least one nonpolar polyolefinic part comprises at least 40 carbon atoms, such as from 60 to 700 carbon atoms. The at least one nonpolar part can be chosen from polyolefins, such as oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene, and 1-octadecene. These polyolefins may or may not be hydrogenated.

Furthermore, as noted above, the at least one polyolefin-derived emulsifying oligomer or polymer used in the composition disclosed herein comprises at least one polar part. The at least one polar part confers amphiphilic properties on the polyolefin derivatives. Thus, the at least one polyolefin-derived emulsifying oligomer or polymer comprising at least one polar part may lower the interfacial (water/oil) tension by at least 10 mN/m when present in an amount of 0.01% by weight, relative to the total weight of the oily phase. For example, the polyolefin comprising a succinic ending sold under the name Lubrizol 2724 by Lubrizol, in an amount of 0.01% by weight, relative to the total weight of the oily phase, lowers the interfacial tension by 15 mN/m at the interface of an aqueous phase comprising a 1% aqueous MgSO₄ solution and an oily phase comprising a mixture of oils (isohexadecane/hydrogenated polyisobutene/volatile silicone in an 8/6/4 ratio).

The at least one polar part of the at least one polyolefin-derived emulsifying oligomer or polymer can be anionic, cationic, nonionic, zwifterionic or amphoteric. The at least one polar part may, for example, comprise polyalkylene glycols, polyalkyleneimines, carboxylic or dicarboxylic acids, or anhydrides orderivatives thereof. The at least one polyolefin-derived emulsifying oligomer or polymer comprising at least one polar part chosen from carboxylic acid polar parts can, for example, result from the reaction between a polyolefin and at least one carboxylic acid or anhydride chosen from maleic acid, maleic anhydride, fumaric acid, itaconic acid, citraconic acid, mesoconic acid and aconitic acid. In one embodiment, the at least one polar part comprises succinic acid or anhydride, ester or amide derivatives thereof, or the corresponding salts of alkali metal, alkaline earth metal or organic ions, or polyoxyethylene.

The polyoxyethylene-derived oligomers or polymers can, for example, be chosen from polyisoprene-polyoxyethylene diblock polymers, poly(ethylene-co-propylene)-polyoxyethylene polymers and mixtures thereof. These polymers are described in the publication by Allgaier, Poppe, Willner and Richter (Macromolecules, 1997, Vol. 30, pp. 1582-1586).

The oligomers or polymers derived from succinic acid or anhydride can be chosen, for example, from the polyolefin derivatives of succinic acid or anhydride disclosed in Patent Nos. U.S. Pat. No. 4,234,435, U.S. Pat. No. 4,708,753, U.S. Pat. No. 5,129,972, U.S. Pat. No. 4,931,110, GB-A-2 156 799, and U.S. Pat. No. 4,919,179. The polyolefin part can comprise, for example, hydrogenated or nonhydrogenated polyisobutylene with a molecular weight ranging from 400 to 5000. In the polyisobutylene comprising a succinic ending thus obtained, the succinic part can be esterified, amidated or in the salt form, that is to say that it can be modified by alcohols, amines, alkanolamines or polyols, or else be found in the form of alkali metal or alkaline earth metal, ammonium or organic base salts, such as diethanolamine and triethanolamine salts. The polyolefins comprising an esterified or amidated succinic ending are reaction products of (a) a polyolefin comprising a succinic ending and of (b) an amine or an alcohol, to form an amide or an ester. As used herein the term “amine” means any type of amine, including alkanolamines. They can be, for example, primary, secondary or tertiary monoamines, it being possible for these amines to be saturated or unsaturated and aliphatic, cycloaliphatic, aromatic or heterocyclic. Furthermore, the alcohols can be mono- or polyalcohols. The monoalcohols comprise primary, secondary or tertiary aliphatic alcohols and phenols. The polyalcohols can be chosen, for example, from aliphatic, cycloaliphatic, aromatic, and heterocyclic polyalcohols. The polyolefins comprising a modified (esterified or amidated) succinic ending and their processes for preparation are disclosed, for example, in U.S. patent no. U.S. Pat. No. 4,708,753.

Examples of polyolefins comprising a succinic ending include polyisobutylenes comprising an esterified succinic ending and salts thereof, for example, diethanolamine salts, such as the products sold under the names Lubrizol 2724, Lubrizol 2722 and Lubrizol 5603 by Lubrizol or the Chemccinate 100AF and 2000 products from Chemron.

Another example of a polymeric surfactant which can be used in the composition disclosed herein is the product of the reaction of maleic anhydride with polyisobutylene, such as the product sold under the name Glissopal SA by BASF.

The at least one polyolefin-derived emulsifying oligomer or polymer may be present in the composition disclosed herein in an amount ranging, for example, from 0.1 to 20% by weight of active material, for example, from 0.2 to 10% by weight, and, further, for example, from 0.5 to 5% by weight, relative to the total weight of the composition. In one embodiment, at least one oligomer or polymer derived from polyolefins may be used.

Examples of the at least one deodorant active salt which can be used include antiperspirant and astringent active pririciples. The at least one deodorant active salt may, for example, be chosen from aluminium and zirconium salts; complexes of zirconium chlorohydrate and aluminium chlorohydrate with an amino acid, such as those disclosed in Patent No. U.S. Pat. No. 3,792,068, commonly known under the name “ZAG complexes” (when the amino acid is glycine).

Mention may, for example, be made, among the aluminium salts, of aluminium chlorohydrate in the activated or nonactivated form, aluminium chlorohydrex, the aluminium chlorohydrex polyethylene glycol complex, the aluminium chlorohydrex propylene glycol complex, aluminium dichlorohydrate, the aluminium dichlorohydrex polyethylene glycol complex, the aluminium dichlorohydrex propylene glycol complex, aluminium sesquichlorohydrate, the aluminium sesquichlorohydrex polyethylene glycol complex, the aluminium sesquichlorohydrex propylene glycol complex, and aluminium sulphate buffered by sodium aluminium lactate.

Mention may, for example, be made, among the aluminium and zirconium salts, of aluminium zirconium octachlorohydrate, aluminium zirconium pentachlorohydrate, aluminium zirconium tetrachlorohydrate, and aluminium zirconium trichlorohydrate.

The complexes of zirconium chlorohydrate and aluminium chlorohydrate with an amino acid are generally known under the name ZAG (when the amino acid is glycine). Mention may be made, among these products, of the aluminium zirconium octachlorohydrex glycine, aluminium zirconium pentachlorohydrex glycine, aluminium zirconium tetrachlorohydrex glycine, and aluminium zirconium trichlorohydrex glycine complexes.

In one embodiment, aluminium chlorohydrate in the activated or non-activated form may be used.

The at least one deodorant active salt disclosed herein can also be chosen from bacteriostatic agents and bactericidal agents, such as quaternary ammonium salts, for example, cetyltrimethylammonium salts and cetylpyridinium salts.

Mention may also be made, among other deodorant active principles in the salt form, of:

zinc salts, such as zinc salicylate, zinc phenolsulphonate, zinc pyrrolidonecarboxylate (more commonly known as zinc pidolate), zinc sulphate, zinc chloride, zinc lactate, zinc gluconate, zinc ricinoleate, zinc glycinate, zinc carbonate, zinc citrate, zinc laurate, zinc oleate, zinc orthophosphate, zinc stearate, zinc silicate, zinc tartrate, zinc lactate, and zinc acetate;

chlorohexidine salts;

polyhexamethylene biguanide salts; and

sodium bicarbonate.

In one embodiment, water-soluble deodorant active salts, such as antiperspirant salts and, for example, aluminium salts may be used.

Deodorant active principles in the salt form can be present in the composition disclosed herein in an amount ranging from 0.1 to 30% by weight, relative to the total weight of the composition and, for example, from 10 to 25% by weight.

The compositions disclosed herein can also comprise at least one additional deodorant active principle. The at least one additional deodorant active principle may be chosen from 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl) urea (Triclocarban) and 3,7,11 -trimethyidodeca-2,5,10-trienol (Farnesol), chlorhexidine, diglycerol monocaprate, diglycerol monolaurate, and glycerol monolaurate.

The cosmetic composition disclosed herein can be provided in a form chosen from a more or less thickened form distributed in a tube or in a twist stick; a roll-on packaged in the ball form; a stick form; and in a spray or aerosol form and can, in this respect, further comprise the ingredients generally used in products of this type which are well known to a person skilled in the art.

The fatty phase in accordance with the water-in-oil emulsions disclosed herein may be present in an amount ranging, for example, from 3 to 80% by weight, relative to the total weight of the emulsion.

The fatty phase generally comprises at least one hydrophobic compound which renders the phase immiscible in water.

The fatty phase, for example, comprises at least one oil chosen from volatile and non-volatile non-silicone hydrocarbon emollient oils.

Mention may be made, among the hydrocarbon emollient oils which can be used, of liquid hydrocarbon derivatives, such as squalane, mineral oils and hydrogenated polyisobutenes and, for example, polydecenes, paraffins and hydrocarbon oils comprising a branched chain which, for example, comprises from 6 to 20, such as from 6 to 18 carbon atoms and which can be chosen, for example, from isohexadecane, isododecane, and isoparaffins; fatty alcohols which are liquid at ambient temperature, such as isostearyl alcohol and octyldodecanol; aliphatic esters of C₃-C₁₈ alcohols with C₃-C₁₈ acids, such as isopropyl myristate, lauryl myristate, isopropyl palmitate, diisopropyl sebacate, and diisopropyl adipate; aromatic esters of benzoic acid with C₁₂-C₁₈ alcohols, and mixtures thereof, such as C₈-C₁₈ alkyl benzoates; ethers of aliphatic fatty alcohols, such as ethers of myristyl alcohol, for example, PPG-3 myristyl ether; (C₁-C₄)alkyl ethers of polyglycols, such as PPG-4 butyl ether; and vegetable oils, such as apricot kernel oil; avocado oil, macadamia nut oil, sunflower oil, olive oil, and soya oil.

The emollient oils disclosed herein can be chosen from volatile and non-volatile silicone oils.

Volatile silicones are defined in a known way as compounds which are volatile at ambient temperature (20-25° C.). They generally exhibit a vapor pressure at 25° C. ranging from 1 or 10 Pa to 2 kPa.

The volatile silicones may be chosen from cyclic and linear volatile dimethylsiloxane silicones, the chains of which comprise from 3 to 9 silicone functional groups.

In one embodiment, the volatile silicones may be chosen from tetramer (D₄), pentamer (D₅), and hexamer (D₆) cyclomethicones.

Nonvolatile silicones are defined in a known way as compounds with a low vapor pressure at ambient temperature. The non-volatile silicones may be chosen from polyalkylsiloxanes, for example, linear polyalkylsiloxanes, such as, the linear polydimethylsiloxanes and dimethicones sold by Dow Corning under the name of “Dow Corning 245 Fluid®”; polyalkylarylsiloxanes, such as, the polymethylphenylsiloxanes sold by Dow Corning under the name of “Dow Corning 556 Fluid®”; and polyether and siloxane copolymers, such as, dimethicone copolyols.

In order to improve the homogeneity of the product, use may be made of at least one suspending agent. The at least one suspending agent may, for example, be chosen from hydrophobic modified montmorillonite clays, such as hydrophobic modified bentonites and hectorites. Mention may be made, for example, of the product Stearalkonium Bentonite (CTFA name) (reaction product of bentonite and of the quaternary ammonium stearalkonium chloride), such as the commercial product sold under the name Tixogel MP 250® by Sud Chemie Rheologicals, United Catalysts Inc., and the product Disteardimonium Hectorite (CTFA name) (reaction product of hectorite and of distearyidimonium chloride), sold under the name of Bentone 38 or Bentone Gel by Elementis Specialities.

The at least one suspending agent may be present in an amount ranging from 0.1 to 5% by weight and, further, for example, from 0.2 to 2% by weight, relative to the total weight of the composition.

The compositions disclosed herein can also comprise, in addition, at least one organic powder.

Mention may be made, among the fillers which can be used in the composition disclosed herein, of organic powders. As used herein, the term “organic powder” means any solid which is insoluble in the medium at ambient temperature (25° C.).

The at least one organic powder may be chosen, for example, from polyamide particles, such as those sold under the names Orgasol® by Atochem; microspheres based on acrylic copolymers, such as those made of ethylene glycol dimethacrylate/lauryl methacrylate copolymer sold by Dow Corning under the name Polytrap®; poly(methyl methacrylate) microspheres sold under the name Microsphere M-100® by Matsumoto and under the name Covabead LH85® by Wacker; powders formed of ethylene/acrylate copolymer, such as those sold under the name Flobeads® by Sumitomo Seika Chemicals; expanded powders, such as hollow microspheres and, for example, the microspheres formed from a terpolymer of vinylidene chloride, of acrylonitrile and of methacrylate sold under the name Expancel® by Kemanord Plast under the references 551 DE 12® (particle size of 12 μm and density 40 kg/m³), 551 DE 20® (particle size of 30 μm and density 65 kg/m³) and 551 DE 50® (particle size of 40 μm), the microspheres sold under the name Micropearl F 80 ED® by Matsumoto; powders formed from natural organic materials, such as powders formed from starch, for example, from crosslinked or noncrosslinked maize, wheat and rice starches, such as the powders formed from starch crosslinked by octenylsuccinic anhydride sold under the name Dry-Flo by National Starch; silicone resin microbeads, such as those sold under the name Tospearl® by Toshiba Silicone, for example, Tospearl 240; and powders formed from amino acids, such as the lauroyllysine powder sold under the name Amihope LL-11® by Ajinomoto.

The aqueous phase of the emulsion comprises water and generally at least one solvent chosen from water-soluble and water-miscible solvents. The at least one solvent chosen from water-soluble and water-miscible solvents comprises short-chain, for example, C₁-C₄, monoalcohols, such as ethanol or isopropanol; or diols or polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether, and sorbitol. In one embodiment, the at least one solvent is chosen from propylene glycol and glycerol.

Water may be present in an amount ranging from 20 to 85% by weight, relative to the total weight of the composition.

The cosmetic composition disclosed herein can also comprise at least one cosmetic adjuvant chosen from softeners, antioxidants, opacifiers, stabilizers, moisturizing agents, vitamins, fragrances, bactericides, preservatives, polymers, thickening agents, and any other ingredient commonly used in cosmetics for this type of application.

Of course, a person skilled in the art will take care to choose this or these optional additional compounds so that the advantageous properties intrinsically attached to the cosmetic composition disclosed herein are not, or not substantially, detrimentally affected by the envisaged addition or additions.

The thickeners, for example, nonionic thickeners, can be chosen from modified and unmodified guar gums and celluloses, such as hydroxypropylated guar gum, cetylhydroxyethylcellulose and silicas, such as Bentone Gel Mio®, sold by NL Industries, and Veegum Ultra®, sold by Polyplastic.

The amounts of these various constituents that can be present in the cosmetic composition disclosed herein are those conventionally used in antiperspirant roll-ons, twist sticks, and creams.

The compositions disclosed herein can also comprise at least one additional agent chosen from structuring and gelling agents for the oily phase, for example, those mentioned herein in the description, such as waxes and/or linear solid fatty alcohols; fatty acids and salts thereof (stearic acid, sodium stearate, 12-hydroxystearic acid); dibenzylidene alditols (DBS); lanosterol; N-acylamino acid derivatives; derivatives of di- and tricarboxylic acids, such as alkyl-N,N′-dialkylsuccinamides (i.e.: dodecyl-N,N′-dibutyl-succinamide); and organopolysiloxane elastomers, such as those disclosed in Patent Application Publication No. WO 97/44010.

The composition disclosed herein can also be pressurized and can be packaged in an aerosol; it then comprises at least one propellant generally used in products of this type which are well known to a person skilled in the art, such as dimethyl ether; volatile hydrocarbons, such as n-butane, propane, isobutene, and mixtures thereof, optionally with at least one chlorinated and/or fluorinated hydrocarbon; mention may be made, among the latter, of the compounds sold by Dupont de Nemours under the names Freon® and Dymel® and, for example, monofluorotrichloromethane, difluorodichloromethane, tetrafluorodichloroethane and 1,1-difluoroethane, sold, for example, under the trade name Dymel 152 A® by Dupont.

The at least one propellant may, for example, be chosen from carbon dioxide gas, nitrous oxide, nitrogen, and compressed air.

The at least one propellant may be present, for example, in an amount ranging from 20 to 85% by weight, relative to the total weight of the composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Concrete but in no way limiting examples illustrating the disclosed embodiments will now be given.

Comparative Stability Tests

The stability over time of various water-in-oil emulsions comprising an antiperspirant active salt (aluminium chlorohydrate) was observed by carrying out several storage cycles comprising periodic rises and falls in temperature.

Each cycle was divided into 4 periods:
6 hours at 20° C.
6 hours of falling to −20° C.
6 hours at −20° C.
6 hours of rising to +20° C.

Each formulation was evaluated macroscopically after each cycle.

| | Example A (prior art; Example 1 |
|---|---|
| Ingredients | of the application DE 10210461) |
| PEG-30 dipolyhydroxystearate | 1.0 g |
| Dicapryl ether | 9.0 g |
| Glycerol | 5.0 g |
| Magnesium sulphate | 0.6 g |
| Aluminium chlorohydrate (50% | 10.0 g |
| aqueous solution) | |
| Fragrance | 1.0 g |
| Water | 73.4 g |
| Stability in cycles | Unstable after 1 cycle |

Example A was prepared according to the process disclosed in the document DE10210461.

| | Example | Example | Example | | |
|---|---|---|---|---|---|
| | B | C | D | | |
| | (outside | (outside | (outside | | Exam- |
| | the | the | the | Example 1 | ple 2 |
| Ingredients | invention) | invention) | invention) | (roll-on) | (roll-on) |
| PEG-30 dipoly- | 1.90 g | 1.90 g | — | — | — |
| hydroxy- | | | | | |
| stearate | | | | | |
| Polyglyceryl-3- | — | — | 1.90 g | | |
| isostearate | | | | | |
| (and) sorbitan | | | | | |
| isostearate | | | | | |
| Polyiso- | — | — | — | 1.90 g | 1.90 g |
| butylene | | | | | |
| comprising an | | | | | |
| esterified | | | | | |
| succinic | | | | | |
| ending, | | | | | |
| diethanolamine | | | | | |
| salt | | | | | |
| (Chemccinate | | | | | |
| 2000) | | | | | |
| Squalane | 2.16 g | 2.16 g | 2.16 g | 2.16 g | 2.16 g |
| Ethylhexyl | 4.65 g | — | — | 4.65 g | — |
| palmitate | | | | | |
| Isohexadecane | — | 4.65 g | 4.65 g | | 4.65 g |
| Cyclohexa- | 2.33 g | 2.33 g | 2.33 g | 2.33 g | 2.33 g |
| siloxane | | | | | |
| Prunus | 1.32 g | 1.32 g | 1.32 g | 1.32 g | 1.32 g |
| armeniaca | | | | | |
| (apricot) kernel | | | | | |
| oil | | | | | |
| Glycerol | 5.00 g | 5.00 g | 5.00 g | 5.00 g | 5.00 g |
| Aluminium | 40.00 g | 40.00 g | 40.00 g | 40.00 g | 40.00 g |
| chlorohydrate | | | | | |
| (50% aqueous | | | | | |
| solution) | | | | | |
| Preservatives | 0.99 g | 0.99 g | 0.99 g | 0.99 g | 0.99 g |
| Water | 41.65 g | 41.65 g | 41.65 g | 41.65 g | 41.65 g |
| Stability in | Unstable | Unstable | Unstable | Stable | Stable |
| cycles | after 1 | after 1 | after 2 | after 10 | after 10 |
| | cycle | cycle | cycle | cycles | cycles |

Formulation Processes:

### EXAMPLES B, C AND D

The fatty phase was homogenized at 75° C. The constituents of the aqueous phase were homogenized separately at ambient temperature with stirring. The aqueous phase was dispersed in the oily phase while stirring with a Rayneri at 600 rpm for 10 minutes and at ambient temperature.

### EXAMPLES 1 AND 2 (ROLL-ON)

Formulation was carried out under the same conditions as for Examples B, C and D but the constituents of the fatty phase were mixed at 45° C. in order to obtain a homogeneous phase.

The water-in-oil emulsions 1 and 2 according to the present disclosure comprising the polyolefin derivative comprising at least one polar part were stable in cycles in comparison with Examples A, B, C and D of the prior art comprising nonionic polyglycerolated or polyoxyethylenated surfactants.

Cosmetic Evaluation:

The water-in-oil emulsions 1 and 2 according to the present disclosure were fresh on application and did not contribute a greasy film during and after the application of the products in comparison with those of the prior art.

### EXAMPLES 3 AND 4

Antiperspirant Creams

| Ingredients | Example 3 | | Example 4 | |
|---|---|---|---|---|
| Polyisobutylene comprising an esterified | 1.92 | g | 1.92 | g |
| succinic | | | | |
| ending, diethanolamine salt (Lubrizol 5603) | | | | |
| Squalane | 2.16 | g | 2.16 | g |
| Ethylhexyl palmitate | — | | 4.65 | g |
| Isohexadecane | 4.65 | g | — | |
| Cyclohexasiloxane | 2.33 | g | 2.33 | g |
| Glycerol | 5.00 | g | 5.00 | g |
| Aluminium chlorohydrate (50% aqueous | 40.00 | g | 40.00 | g |
| solution) | | | | |
| Prunus armeniaca (apricot) kernel oil | 1.32 | g | 1.32 | g |
| Preservatives | 0.99 | g | 0.99 | g |
| Water | 41.63 | g | 41.63 | g |
| Viscosity at 24° C. (spindle 3, 200 s⁻¹) | 5.1 | Pa · s | 4.5 | Pa · s |

Formulation Process:

The fatty phase was homogenized at 45° C. The constituents of the aqueous phase were mixed separately with stirring at ambient temperature until homogeneous. The aqueous phase was dispersed in the oily phase with stirring using a Rayneri at 600 rpm for 10 minutes.

The formulations were in the form of a soft, smooth and glossy cream. The formulations were stable from 4 to 45° C. for 2 months.

### EXAMPLE 5

Antiperspirant Aerosol:

| Ingredients | Example 5 | |
|---|---|---|
| Polyisobutylene comprising an esterified succinic ending, | 0.96 | g |
| diethanolamine salt (Lubrizol 5603) | | |
| Squalane | 1.08 | g |
| Isohexadecane | 2.33 | g |
| Cyclohexasiloxane | 1.17 | g |
| Prunus armeniaca (apricot) kernel oil | 0.66 | g |
| Cyclopentasiloxane (and) Disteardimonium Hectorite (and) | 1.50 | g |
| Denatured alcohol | | |
| Glycerol | 2.50 | g |
| Aluminium chlorohydrate (50% aqueous solution) | 20.00 | g |
| Water | 19.81 | g |
| Isobutane | 50.00 | g |

Formulation Process:
Preparation of the Dispensable

The process for manufacturing the dispensable was the same as that of the examples above.

Pressurization

The dispensable was pressurized with isobutane according to conventional techniques.

The white residue phenomenon after application with the aerosol according to the present disclosure was subsequently observed and compared with the Clearex® formulation from Unilever, known to be not very whitening on application.

Approximately 0.3 g of dispensable (amount without propellant) was sprayed, at a distance of 15 cm, on a bioskin substrate (black plate) sold by Maprecos S.A.

The substrate was subsequently placed in a light box. The area treated was filmed for 4 hours using a Microvision black and white camera at the rate of 1 photograph every 10 seconds. The shade of grey observed on the substrate as a function of the time was measured and calculated using a software program.

This shade of grey represented the white residue on the substrate after application. The higher the value, the whiter the shade.

The values of greyness measured for each aerosol are represented in the following table:

| Formulations | Level of greyness after 1 hour |
|---|---|
| Example 5 (invention) | 65 |
| Clearex ® aerosol | 73 |

The formulation was less whitening than the market reference.

### EXAMPLE 6

Antiperspirant Stick:

| Ingredients | Example 6 | | Example 7 | |
|---|---|---|---|---|
| Polyisobutylene comprising an esterified succinic | 1.90 | g | 1.90 | g |
| ending, diethanolamine salt (Chemccinate 2000) | | | | |
| Isohexadecane | 10.60 | g | 10.60 | g |
| Hydrogenated castor oil | | | 3.00 | g |
| Stearyl alcohol | 13.00 | g | 11.00 | g |
| Aluminium chlorohydrate (50% aqueous solution) | 40.00 | g | 40.00 | g |
| Glycerol | 5.00 | g | 5.00 | g |
| Water | 29.5 | g | 28.5 | g |

Formulation Process:

The fatty phase was melted, at approximately 80° C., until the medium was homogeneous. The constituents of the aqueous phase were mixed separately with stirring at ambient temperature until homogeneous. The aqueous phase was added dropwise to the oily phase (20 g of water/minute) while stirring with a Rayneri at 400 rpm. The mixture was left stirring with the Rayneri for 20 minutes at 1000 rpm. The emulsion was cast, under hot conditions, into sticks and was rapidly cooled to +4° C. Casting was carried out approximately at 5° C. above the softening point of each stick.

1. A cosmetic composition comprising,
(A) at least one deodorant active salt; and
(B) at least one polyolefin-derived emulsifying oligomer or polymer comprising at least one polar part,
wherein the composition is in the form of a water-in-oil emulsion.

2. The composition according to claim 1, wherein the at least one polyolefin-derived emulsifying oligomer or polymer comprising at least one polar part further comprises at least one nonpolar polyolefinic part comprising at least 40 carbon atoms.

3. The composition according to claim 2, wherein the at least one polyolefin-derived emulsifying oligomer or polymer comprising at least one polar part further comprises at least one nonpolar polyolefinic part comprising from 60 to 700 carbon atoms.

4. The composition according to claim 2, wherein the at least one nonpolar polyolefinic part is chosen from oligomers, polymers and copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene, and 1-octadecene.

5. The composition according to claim 1, wherein the at least one polyolefin-derived oligomer or polymer comprising at least one polar part lowers the interfacial tension by at least 10 mN/m when it is present in an amount of 0.01% by weight, relative to the total weight of oily phase.

6. The composition according to claim 1, wherein the at least one polar part is anionic, cationic, nonionic, zwitterionic, or amphoteric.

7. The composition according to claim 1, wherein the at least one polar part comprises polyalkylene glycols, polyalkyleneimines, carboxylic and dicarboxylic acids, and anhydrides and derivatives thereof.

8. The composition according to claim 1, wherein the at least one polar part comprises polyoxyethylene, succinic acid, and anhydride and derivatives thereof.

9. The composition according to claim 1, wherein the at least one polyolefin-derived oligomer or polymer comprising at least one polar part results from the reaction between a polyolefin derivative and at least one acid chosen from maleic acid, maleic anhydride, fumaric acid, itaconic acid, citraconic acid, mesaconic acid, aconitic acid, and derivatives thereof.

10. The composition according to claim 1, wherein the at least one polyolefin-derived oligomer or polymer comprising at least one polar part is a polyisobutylene comprising an optionally modified succinic ending.

11. The composition according to claim 10, wherein the at least one polyolefin-derived oligomer or polymer comprising at least one polar part is a product of a reaction of maleic anhydride with polyisobutylene.

12. The composition according to claim 1, wherein the at least one polyolefin-derived oligomer or polymer is present in an amount ranging from 0.1 to 20% by weight of active material, relative to the total weight of the composition.

13. The composition according to claim 1, wherein the at least one deodorant active salt is chosen from antiperspirant salts.

14. The composition according to claim 13, wherein the antiperspirant salts are chosen from aluminium and zirconium salts; and complexes of zirconium chlorohydrate and of aluminium chlorohydrate with an amino acid.

15. The composition according to claim 14, wherein the antiperspirant salts are chosen from aluminium chlorohydrate in the activated or nonactivated form, aluminium chlorohydrex, the aluminium chlorohydrex polyethylene glycol complex, the aluminium chlorohydrex propylene glycol complex, aluminium dichlorohydrate, the aluminium dichlorohydrex polyethylene-glycol complex, the aluminium dichlorohydrex propylene glycol complex, aluminium sesquichlorohydrate, the aluminium sesquichlorohydrex polyethylene glycol complex, the aluminium sesquichlorohydrex propylene glycol complex, and aluminium sulphate buffered by sodium aluminium lactate.

16. The composition according to claim 14, wherein the antiperspirant salts are chosen from aluminium zirconium octachlorohydrate, aluminium zirconium pentachlorohydrate, aluminium zirconium tetrachlorohydrate, and aluminium zirconium trichlorohydrate.

17. The composition according to claim 14, wherein the antiperspirant salts are chosen from complexes of zirconium chlorohydrate and aluminium chlorohydrate with glycine (ZAG).

18. The composition according to claim 16, wherein the antiperspirant salts are chosen from aluminium zirconium octachlorohydrex glycine, aluminium zirconium pentachlorohydrex glycine, aluminium zirconium tetrachlorohydrex glycine, and aluminium zirconium trichlorohydrex glycine complexes.

19. The composition according to claim 1, wherein the at least one deodorant active salt is chosen from bacteriostatic agents and bactericidal agents.

20. The composition according to claim 19, where the at least one deodorant active salt is chosen from quaternary ammonium salts.

21. The composition according to claim 20, wherein the at least one deodorant active salt is chosen from cetyltrimethylammonium salts and cetylpyridinium salts.

22. The composition according to claim 1, wherein the at least one deodorant active salt is chosen from:
zinc salts;
chlorohexidine salts;
polyhexamethylene biguanide salts; and
sodium bicarbonate.

23. The composition according to claim 22, wherein the zinc salts are chosen from zinc salicylate, zinc phenolsulphonate, zinc pyrrolidonecarboxylate, zinc sulphate, zinc chloride, zinc lactate, zinc gluconate, zinc ricinoleate, zinc glycinate, zinc carbonate, zinc citrate, zinc laurate, zinc oleate, zinc orthophosphate, zinc stearate, zinc silicate, zinc tartrate, zinc lactate, and zinc acetate.

24. The composition according to claim 1, wherein the at least one deodorant active salt is water-soluble.

25. The composition according to claim 24, where the water-soluble deodorant active salt is aluminium chlorohydrate in the activated or nonactivated form.

26. The composition according to claim 1, wherein the at least one deodorant active salt is present in an amount ranging from 0.1 to 30% by weight, relative to the total weight of the composition.

27. The composition according to claim 26, wherein the at least one deodorant active salt is present in an amount ranging from 10 to 25% by weight, relative to the total weight of the composition.

28. The composition according to claim 1, further comprising at least one additional deodorant active principle which is not in the salt form.

29. The composition according to claim 28, wherein the at least one additional deodorant active principle is chosen from 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl) urea (Triclocarban) and 3,7,11-trimethyidodeca-2,5,10-trienol (Farnesol), chlorhexidine, diglycerol monocaprate, diglycerol monolaurate, and glycerol monolaurate.

30. The composition according to claim 1, wherein the composition is provided in a form chosen from a thickened form distributed in a tube or in a twist stick; a roll-on packaged in a ball form; a stick form; and a spray or aerosol form.

31. The composition according to claim 1, wherein the oily phase comprises at least one volatile or non-volatile non-silicone hydrocarbon emollient oil.

32. The composition according to claim 1, further comprising at least one suspending agent.

33. The composition according to claim 1, further comprising at least one organic powder.

34. The composition according to claim 1, further comprising at least one cosmetic adjuvant chosen from waxes, softeners, antioxidants, opacifiers, stabilizers, moisturizing agents, vitamins, fragrances, bactericides, preservatives, polymers, and thickening agents.

35. The composition according to claim 1, further comprising at least one structuring or gelling agent for the oily phase.

36. The composition according to claim 1, wherein the composition is pressurized and packaged in an aerosol and wherein the composition further comprises at least one propellant.

37. A cosmetic process for treating perspiration comprising applying, to the surface of the skin, an effective amount of at least one composition comprising,
(A) at least one deodorant active salt; and
(B) at least one polyolefin-derived emulsifying oligomer or polymer comprising at least one polar part,
wherein the composition is in the form of a water-in-oil emulsion, and
wherein the at least one deodorant active salt and the at least one polyolefin-derived emulsifying oligomer or polymer are present in a combined amount sufficient to treat perspiration.

38. A cosmetic process for treating body odors related to perspiration comprising, applying, to the surface of the skin, an effective amount of at least one composition comprising,
(A) at least one deodorant active salt; and
(B) at least one polyolefin-derived emulsifying oligomer or polymer comprising at least one polar part,
wherein the composition is in the form of a water-in-oil emulsion, and
wherein the at least one deodorant active salt and the at least one polyolefin-derived emulsifying oligomer or polymer are present in a combined amount sufficient to treat body odors related to perspiration.

39. A method for stabilizing a water-in-oil emulsion comprising at least one deodorant active salt comprising including in a water-in-oil emulsion comprising at least one deodorant active salt, at least one polyolefin-derived emulsifying oligomer or polymer comprising at least one polar part wherein the at least one polyolefin-derived emulsifying oligomer or polymer is present in an amount sufficient to stabilize the water-in-oil emulsion.

40. A method for improving at least one sensory property on skin comprising
applying, to the surface of the skin, an effective amount of at least one composition comprising at least one polyolefin-derived emulsifying oligomer or polymer comprising at least one polar part and at least one deodorant or antiperspirant active salt,
wherein the composition is in the form of an water-in-oil emulsion and
wherein the at least one polyolefin-derived emulsifying oligomer or polymer is present in an amount sufficient to improve at least one sensory property on the skin upon application.
